(19) United States
(12) Patent Application Publication
(10) Pub. No.: US 2011/0040358 A1
(43) Pub. Date: Feb. 17, 2011

(54) Portable Semiconductor Diode Laser for Medical Treatment

(75) Inventors: David M. Bean, Middleton, MA (US); Donald C. Freeman, Burlington, MA (US)

Correspondence Address: HOUSTON ELISEEVA, 420 BEDFORD ST, SUITE 155, LEXINGTON, MA, 02420, US

(73) Assignee: SEMINEX CORPORATION, Peabody, MA (US)

(21) Appl. No.: 12/920,947

(22) PCT Filed: Mar. 3, 2009
(86) PCT No.: PCT/US09/01350

§ 371 Date: Sep. 3, 2010

Related U.S. Application Data
(60) Provisional application No. 61/067,915, filed on Mar. 3, 2008.

(51) Int. Cl. A61N 5/06

(52) U.S. Cl. 607/89

(57) ABSTRACT

Semiconductor lasers, in which the laser diode output is used directly for medical treatment, can be compact, lightweight and efficient. Lasers operating in the relatively “eye-safe” window of about 1350-1600 nm can be used to treat wounds and diseases of the skin and other organs, for example to cut, ablate or coagulate bodily tissues and fluids. Because the wavelength is eye-safe, the treatment can be through-space, and does not require the use of a fiber optic containment system. The device need not directly contact tissue. Such devices can be battery operated, are portable and self-contained, and thus are suitable for uses that are not confined to medical facilities, including use in emergency situations and in military operations.

### PRIORITY

This application claims the benefit of the priority of U.S. Provisional application 61/067915, which was filed in the United States Patent and Trademark Office on Mar. 3, 2008, and which is incorporated in its entirety by reference where permitted.

### BACKGROUND

Ever since their invention, lasers have been proposed for use, or used, in medical treatment. Early US patents for laser surgical treatment include, for example, U.S. Pat. No. 3,456,651 to Smart, in which a ruby laser beam is used to coagulate the retina; U.S. Pat. No. 3,865,113, Sharon et al, using a laser beam as a scalpel; and U.S. Pat. No. 4,273,127, Auth et al, using a laser for incisions via a flexible fiber waveguide. These earlier systems use large, complex, and typically fixed laser systems, largely for cutting.

More recent systems are typically more complex, and increasingly use infrared (IR) light for treatment. Examples include EP 0 198 959, Dwyer et al, using a beam from a multimode YAG laser at 1000 or 1300 nm to respectively cut and cauterize a site. The beam is directed to the site via an optical fiber. Raven et al, U.S. Pat. No. 4,917,486, use a dichroic beam splitter to focus both a visible tracking beam and an infrared laser diode beam at 800-900 nm on a site for photocoagulation of lesions of the retina. Complex optics are required to allow the lesion to be seen in visible light while the infrared light is used to coagulate the lesion. The coagulation wavelength is selected to be in the near IR (about 800 nm) because absorption of radiation by the retina is said to decline dramatically with increasing wavelength. Amirkhanian et al, U.S. Pat. No. 5,349,590, use a much longer wavelength (ca. 3000 nm, or 3 microns) from a Er:YAG laser, which is absorbed by most materials and must be delivered to the target site by a waveguide.

Current systems tend to be more portable. Colles et al., EP 1 279 375, describe a generic portable medical laser system which is powered by a battery, delivering energy through a fiber optic “laser applicator”, and which has a safety means to avoid eye injury, so that the laser cannot be operated without depressing a foot pedal or other control. Wavelengths used range from 470 to 1000 nm, i.e., visible and near IR (infra-red). At similar wavelengths, a pressure sensor in the beam emittere is used by Zenzie et al, US 2008/0319430, to prevent exposure of the eye to a medical laser beam.

Keipert et al, U.S. Pat. No. 5,553,629, describe using an IR semiconductor laser diode to treat battlefield wounds, with a fiber optic cable between the diodes and the point of emission of radiation. Wavelengths are typically about 800 nm; the device can also be used as an IR flashlight, with suitable goggles. Johnson et al, U.S. Pat. No. 5,147,389, describes using IR laser diodes emitting at 700-840 nm via a fiber to photocoagulate tissue on the retina, with a low power red HeNe laser beam merged into the IR laser beam to provide positioning information. Lenses may be used to shape the IR laser beam profile to be essentially circular. Mucheryan, U.S. Pat. No. 4,808,789, used a laser diode to increase the efficiency of pumping of a “laser rod” or fiber, such as Nd-YAG, Er-YAG, ruby, or alexandrite. Laser diodes include GaAs (ca. 800 nm), GaAlAs 750-900 nm), InGaAsP (1100-1800 nm), and GaInAlAs. The diode lasers are focused on the lasing rod from the side or axially. Axial transmission can be via a fiber leading from the diode to the lasing rod.

Anderson et al, EP 1 011811 B1, uses any of several lasers, as well as incoherent sources, for treating wrinkled skin by selectively shrinking collagen fibers in the lower dermis. They use sources in the range of 1.3 to 1.8 micron (1300-1800 nm), including Nd:YAG at 1330 and 1440 nm, and Er:glass at 1540 nm. All of these sources are pumped. The laser pulses are delivered to the skin via a fiber optic and through a cooling zone to minimize epidermal damage. The Er:glass laser cited by Anderson is described in Sinovsky, EP 0 214 712 B1, where it “eliminates retinal eye damage” (col. 2 line 39 ff.) , but is not suitable for removing atherosclerotic plaque. The laser systems used by Sinovsky instead use YAG-type lasers including variously doped YAG and YLF lasers in the range of 1500-2200 nm to remove plaque; all are flash-pumped.

### SUMMARY OF THE INVENTION

None of these systems is entirely satisfactory for mobile use in the field, or other non-controlled environment, to coagulate, cauterize, cut, ablate, or otherwise treat tissues of the body. Moreover, exploitation by these systems of the favorable conditions for both safety and efficacy created by operating in the 1350-1600 nm water absorption band is minimal. In particular, the existing systems that do use the 1350-1600 region are optically pumped (in contrast to electrically driven devices, such as semiconductor lasers), and so are complex, expensive, and generally require cooling. None of these optically pumped systems are suitable for portable or field use, nor are they inexpensive enough to be potentially disposable.

In one aspect, the invention comprises a hand-held portable semiconductor diode laser with a wavelength in the range of about 1300 to about 1700 nm, preferably about 1350 to about 1600 nm, having sufficient power for direct treatment of wounds and diseases of the skin and other tissue. A preferred material for the laser is InGaAsP, but other primary diode emitters in the general wavelength band of 1350-1600 nm could be suitable. Treatment with the diode laser can include coagulation, cauterization, cutting, surface heating, and ablation, and other applications requiring locally intense energy transfer. Treatment with the instrument of the invention can also include treatment of diseases of the skin, and other lower power uses. In one aspect, the device can be used in battlefield or emergency use, and in another aspect, in conventional surgical or medical use.

An important feature of the invention is that the laser is coupled through space to the target tissue, and the laser beam does not require confinement in a fiber optic. This simplification is made possible by improved packaging, and by the selection of wavelength. The optical design and improved packaging also simplify thermal management of the diode laser, which at useful power levels does not require liquid cooling. Moreover, in contrast to other portable lasers, the laser of the invention emits in the water-absorption region of the spectrum, particularly in the 1350-1600 nm region, which is relatively “eye-safe” (protects the retina of users even if eye safety gear fails), while being readily absorbed by tissue.

Packaging of the system is simple. A housing contains and supports one or more semiconductor diode lasers, emitting in the range of about 1300-1700 nm, preferably about 1350-1600 nm, more preferably in the 1400-1550 nm range. The optical beams emitted by one or more semiconducter laser diodes are typically in contact with at least one lens, for example a cylindrical, ball or micro-optic lens, for controlling beam dispersion. Optionally, additional focusing means are provided, either as part of the cylindrical lens or as a separate, optionally adjustable lens. The laser may be powered by a battery (optionally rechargeable), or can be connected to a remote battery or portable power supply, or to an electric grid. A stand may be provided for positioning the lens or the housing at one or more specific distances from a tissue surface. Preferably, the device includes a visible light source to assist in visualizing the site of operation and in aiming the invisible IR beam at the target.

In one aspect, the invention comprises a portable semiconductor diode laser system for medical treatment, the system comprising a semiconductor laser diode emitting in the infrared between about 1300 and about 1700 nm, preferably about 1350 to about 1600 nm, more preferably between about 1400 to about 1550 nm; a power supply to drive said diodes; and a packaging suitable for rendering the device portable, for example by holding in the hand or placing in a simple strand, to administer laser energy to a patient, said laser energy being effective for coagulation, cautery, cutting, localized heating, ablation of tissue, or treatment of skin diseases, or in functional equivalents of these processes by whatever name. In the system, a beam is conducted from the emitting diode to the site of treatment at least in part through space, i.e. at least in part without the use of fiber optic conductors or other waveguides. In a preferred embodiment, the laser diode is a InGaAsP diode.

The laser-based system may be used in a treatment selected from coagulation, cauterization, ablation and cutting, treatment of skin disease, surface treatment of tissue, and functional equivalents thereof. The system may further comprise an aiming system using visible light to assist the operator in directing the infrared laser beam to the site of treatment. The emitted laser beam may be parallel or slightly diverging, or it may be focused to a point and afterwards diverge. A lens or lenses may be used to form a focal point, or to adjust the location of a focal point with respect to the diode laser.

The system may have an associated stand to position the diode laser source at an appropriate distance from the patient. The stand may maintain a fixed distance between the housing of the laser source and the site of operation on the patient, or the stand can be set to provide one of two or more preselected distances between the laser source and the site of operation on the patient, or the distance between the laser and the patient can be varied continuously by adjustment of the stand. The stand is preferably transparent to visible light, is optionally non-transparent to 1300-1700 nm infrared light, and may be open (non-continuous), consisting for example of a ring or feet capable of resting on a tissue surface, and rods or other connectors creating a fixed distance between the ring or feet and the housing of the laser.

In another embodiment, no provision is made for a spacing device such as a stand. The housing in this embodiment is preferably thin and elongated, and the surgeon or other medical or paramedical operator can handle the inventive device as if it were a scalpel, or a flashlight, controlling the degree of cutting by watching the effect of the laser light on the tissue as revealed by the tissue or by an illuminating or pointing visible beam, optionally connected to the diode laser device or housing.

In one embodiment, the laser beam comes to a focus, and the locus of the focal point can be adjusted by at least one of an adjustment of a stand, an adjustment of an optical focusing means provided with a stand, an adjustment of a focusing means in a device without a stand, and by free hand movement by the operator. In the system, a power supply for the device is provided by one or more of a rechargeable battery, a non-rechargeable battery, a wired connection to a low voltage power source, and a wired connection via a transformer to a high voltage power source. If the power provided is sufficient, a wireless power supply can be used.

The system is used to carry out at least one medical procedure. The procedure may be selected from the coagulation of blood, the cauterization of wounds, the ablation of tissue, the cutting of tissue, the treatment of tissue surfaces, and the treatment of skin, especially skin diseases. Other procedures that can be performed with a diode laser device of the invention are likewise available for use.

Thus, the system provides a method for conducting at least one procedure, where the method comprises a first step of providing a portable semiconductor diode laser operating in the 1300-1700 nm band, preferably in the 1350-1600 nm band, more preferably in the 1400-1550 nm band, together with a power source, in a portable housing.

Portability requires low weight and volume. The laser with its local power source, such as a battery or power supply can be characterized as weighing less than about 5 kg, preferably less than 2 kg, optionally less than 1 kg. The laser without the power supply or battery can weigh less than 2 kg, preferably less than 1 kg, optionally less than 200 g. The system can provide a focus for the laser, and an optional variable position lens to control the location of the focus.

As a second step, the laser beam is applied to tissue in need of treatment, to cut (including incise and excise), coagulate, cauterize, or otherwise treat the tissue. The laser beam is applied to tissue primarily through space, rather than through an optical fiber. Any fiber optic in the beam path is limited to a portion of the total beam path, such as less than 50% of the physical distance from the laser diode to the site of treatment, and preferably less, such as less than 33%, or such as less than 25%, or less than 10%. More preferably, the beam is transmitted from the laser diode to the tissue site of treatment through space, with the optional assistance of lenses, but without optical fiber or other optical waveguides.

In another aspect, the method may include providing a stand to control the distance between the laser and the tissue to be affected by said medical procedure; adjusting said stand if required to achieve the best distance for the particular procedure desired; and activating said diode laser for sufficient time to conduct said selected procedure.

In another aspect, the tissue site that is treated is on the exterior of the body. Alternatively, the site may be internal. Optionally, a portable semiconductor diode laser may carried on an endoscope or catheter. The tissue site may accessed through a natural body passage or opening, or an opening in a passage, to reach a site of operation. The natural body passage may be selected from one or more of an artery, a vein, a nasal passage, the alimentary and gastrointestinal tracts, the pulmonary tract, the milk ducts, the urinary tract, the female reproductive tract and the male reproductive tract.

In another aspect, in the system or in a method of its use, the emitted optical power is preferably greater than 1 watt. In another aspect, the laser is preferably pulsed while in use to aid in management of the generated heat.

### BRIEF DESCRIPTION OF THE FIGURES

FIG. 1 shows the general structure of a portable medical device of the invention.

FIG. 2 shows an alternative device structure, in which the laser beam is focused.

FIG. 3 shows a device with means for adjusting the focus.

FIG. 4 shows a device with a stand for fixing the distance from the device to the patient.

FIG. 5 shows a device with a stand with a laser beam that comes to a focus.

FIG. 6 shows schematically how focus could be adjusted.

FIG. 7 shows the absorption coefficient of water in the visible through infrared areas of the spectrum.

FIG. 8 shows schematically the details of assembly of a preferred laser for use in the system and methods of the invention.

FIG. 9 shows a laser assembly in a suitable heat sink assembly.

### DETAILED DESCRIPTION OF THE INVENTION

As used herein, the phrase “semiconductor diode laser”, or simply “diode laser”, means a semiconductor device which emits photons in a laser-like manner, i.e. as generally coherent and monochromatic pulses or continuous beams. These lasers are distinguished from lasers that are pumped by external light sources (which may themselves be lasers, including diode lasers). Diode lasers are typically and preferably capable of being mass-produced by standard chip fabrication methods. They may have one or more lenses or other focusing or beam-shaping means included in their fabrication and/or packaging, to control the size and divergence or convergence of the beam when it is emitted from the laser package. Suitable diode lasers are described in our co-pending application US 2007/0002915 and its equivalents, but other laser systems may be useful in the invention.

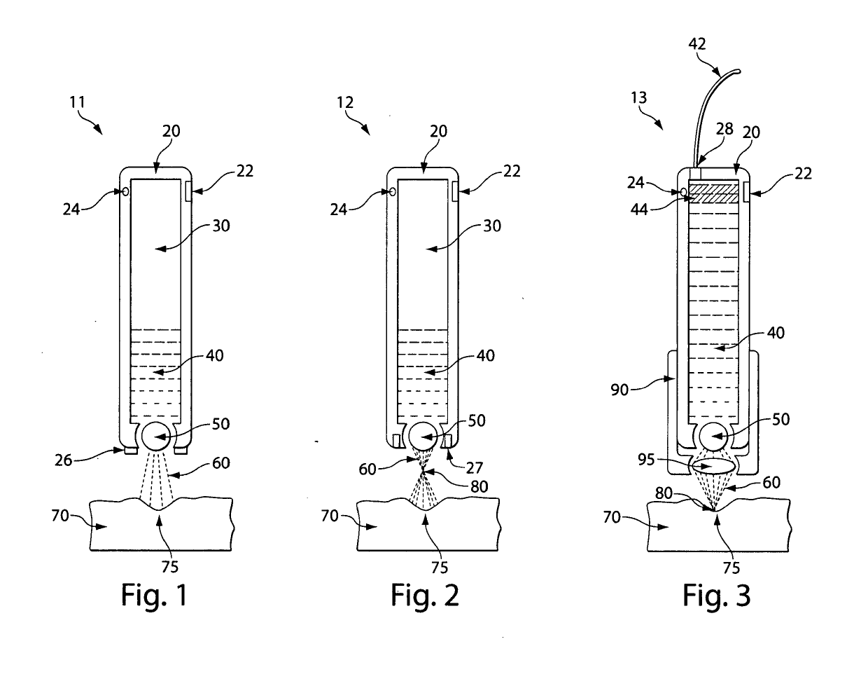

FIG. 1 shows a first version of the device of the invention. (The Figures herein showing the device are generally schematic, to show functional parts and their relationships. The proportions and detail of an actual device will likely be different.) The device 11 has a housing 20 which carries a power supply 30, which may be one or more of a rechargeable battery, a non-rechargeable battery, a wired connection to a low voltage power source (e.g. a vehicle battery), and a wired connection via a transformer or other converter to a high voltage power source (e.g. AC current.) A battery is preferred, and can be removable and/or rechargeable, or disposable.

The battery 30 is shown as being in the housing with laser diodes 40, but the battery or other power supply may also be located elsewhere, for example on the back of the device, or on a belt pack and connected to the laser diodes via a wire.

Housing 20 may have ancillary devices including a power switch 22, an indicator of laser activity 24, and one or more visible light sources 26 to illuminate the field of the procedure. The visible light source 26 may be one or several discrete sources, for example conventional LEDs (light emitting diodes), or could be a strip source encircling the end of the housing 20.

The laser diode 40 is typically mounted integrally with a heat sink (which is not separately numbered here; see below). A heat sink is required to dissipate heat from the device and prevent it from overheating and failing in operation. The preferred heat sink material is diamond, followed by copper, beryllium oxide and aluminum nitride. When the heat sink is also to be electrically conductive, copper is generally preferred.

The heat sink may have ribs or fins, and/or other devices and/or ventilation, to dissipate heat generated by the laser, and heat dissipation may also be promoted by an electrical fan. The heat sink will account for much of the device's size. As illustrated, the laser system and heat sinks, numbered 40 in the drawings, can occupy over 25%, more typically 50% or more, of the interior volume of the laser device. In preferred embodiments, the entire instrument will be about the size and weight of an electric razor, or less.

The battery, or other power source, powers a semiconductor laser diode 40 or an array of such lasers. The laser diode 40 may be focused by a lens system 50, which may be a cylindrical lens, seen end-on in FIG. 1-5, or a spherical or other lens. The size of the device is not critical, as long as it is readily portable, preferably weighing less than 5 kg including battery, and more preferably less than 2 kg. With a small battery, or a remote battery or power supply coupled to the diode laser, a size range of 0.5 to 2 cm diameter and 10-20 cm in length is possible—i.e., a size in the general size range of a large pen or pencil—in effect, a laser scalpel. With a larger battery, the device may be in the general size range of a flashlight, as shown below.

When there is no battery pack in the housing 20, and power is supplied via an electric wire (or wirelessly) to the laser diodes 40, and the optional visible light source 26, then the housing, with lasers, lens and optionally focusing or positioning means, may weigh less than 100 g, and may weigh in some embodiments less than 30 g (about 1 oz), which is typical of the weight of a metal ball point pen. The preferred light-weight embodiments will feel natural to practitioners experienced with scalpels and similar conventional instruments. The outer surface of the housing will have a texture and shape adapted to facilitate holding the instrument in the hand during its use.

A beam 60 is emitted through the lens and impacts on the patient's tissue 70 at a selected site 75. In the embodiment pictured, the beam 60 is slightly diverging. Depending on laser power, such a beam may be able to coagulate blood at the site, or lightly cauterize a wound. The progress of the procedure can be visually observed by the operator of the device, using ambient light and/or light from an illuminator 26, and the device will be turned off when the objective is accomplished.

FIG. 2 shows a similar pen-like or flashlight-like device 12 having the same parts as the device 11 of FIG. 1, except that the lens 50 has been constructed and placed so that the laser beam 60 converges to a focal point at focus 80. (Also, an alternative mounting 27 of an illuminating light is shown.) Clearly the power density at the focus 80 will be considerably higher than in FIG. 1, and this instrument, at a given amount of laser power output, will be more capable of incising and ablating tissue than a non-focusing device. In this arrangement, the operator can vary the power applied to tissue by controlling the distance of the housing 20 from the patient. As illustrated, the focal point 80 is slightly above the tissue surface.

FIG. 3 shows a similar device 13. Device 13 has no battery pack 30, and instead has a power and control cable 42 connecting to a plug 28, and a small on-board electronics and control package 44. Cable 42 is connected to a source of electric power. Device 13 also has, in addition to a first lens 50 associated with the diode array 40, here illustrated as emitting a diverging beam, a second lens 95, mounted in a sliding carrier 90 which slides along the housing 20. Carrier 90 can be held in place by a friction fit, or by more complex (and more precise) means, described below. Depending on the position of the carrier 90, the beam 60 can be focused at varying distance from the lower end of the housing 20. As illustrated, the focus 80 is at the surface of a site 75 of the tissue 70.

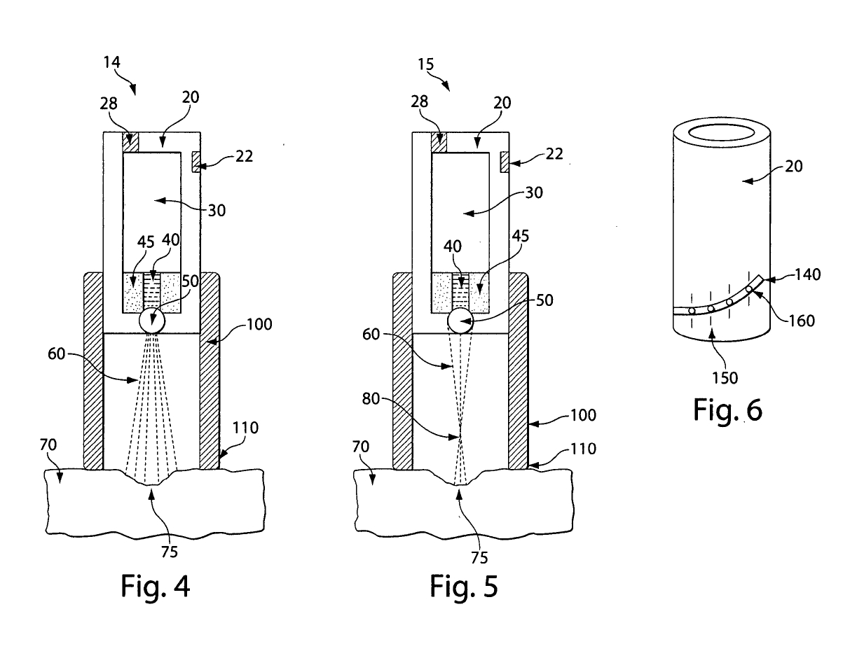

In another embodiment, shown in FIG. 4, a device 14 having a housing 20, a switch 22 and a power connection 28, optionally contains a battery pack or power transformer 30, a laser assembly 40, an optional electronics package 45, and a lens 50. The device 14 is further provided with a stand 100. The stand, in one embodiment, slides from a first non-deployed (retracted) position, not illustrated, in which it surrounds and is approximately co-extensive with the housing 20, to the illustrated deployed position, in which the distal end 110 of the stand 100 contacts the patient, thereby setting the distance between the lens 50, and the site of treatment 75 on the patient's body 70. For example, site 75 may be a wound.

The stand 100, when it is deployed, may expose an on-off switch 22 on housing 20. The stand 100 when undeployed may be held in place on the housing at one end or the other (or both) by simple mechanisms (not illustrated), such as a set screw, a latch, a cap, a limiting ring, one or more detents for a protrusion, a friction fit, and the like. Means are provided to retain the stand in its deployed state. For example, the stand 100 could be held in the undeployed state by plastic caps, said plastic caps fitting onto the stand at each end by a friction fit or a screw fit; and then the stand 100 can be retained in the deployed state by a groove near the bottom of the housing, into which a lip of the stand fits and catches (not illustrated), or it could be retained by a set screw or other device.

Preferably, at least the externally-exposed parts of the system can be sterilized. Sterilization may be limited to certain procedures, which the materials of the device will be selected to withstand. Conventional sterilization means are preferred, which include, among others, autoclaving, treatment with chemical sterilizing agents (for example ethylene oxide), and other procedures known for device sterilization. For example, the battery pack or power supply can be removed from the housing; the housing, the optional stand, and other durable components can be sterilized; and the system can be reassembled under sterile conditions, and sealed in a sterile pouch.

Moreover, because the lower edge 110 of the stand 100 (when provided) is a part that contacts the patient, multiple sterile stands can be provided; or sterile cloths with one or more holes for the laser beam can be draped over the area to be treated. It may be possible to make the system or its parts sufficiently cheaply that the system or parts of it can be discarded after treatment of a patient. For example, in the pen-like design of FIG. 1, and especially where a remote power supply is connected with a wire, the part of the system that contacts the patient—in essence the housing 20—can be discarded at the end of the procedure.

The stand 100, when provided, can assist a less-experienced operator in delivering the appropriate intensity of energy to a site, or can assist the operator in maintaining the relationship of a beam focus to a tissue surface. This is shown in FIG. 5, where the device 15 is essentially the same as the device in FIG. 4, except that the lens 50 is arranged so that the beam 60 has a focal point 80 near the tissue surface 75. The presence of the stand 100 allows accurate control of the distance of the focal point 80 from the tissue surface, which in turn allows better control of the procedure, which for example could be a cauterization procedure, or other procedure.

When an optical system that produces a beam with a focal point is provided, for example such as focal point 80 in FIG. 5, it can be useful to allow deployment of the stand to be variable. In a first embodiment, shown in FIG. 6, the stand (not shown) could be connected to the housing 20 by a spiral groove 140, similar to connections in a zoom lens, so that by rotating the stand relative to the housing 20, the distance from the lens 50 of the distal end 110 of the stand (as seen in FIG. 5, for instance), and hence the patient surface 75, can be controlled. A series of marks 150 along the groove 140 can designate the proper location, again after the manner of a zoom lens. Means for fixing the relative rotational positions of the stand 100 and the housing 20 are preferably provided.

Any of a variety of conventional mechanical devices can be used for such a purpose. For example, a spring-loaded pin on the stand 100 (not illustrated) could fit into one of several holes 160 on the housing 20, in the groove 140 (as illustrated in FIG. 6) or elsewhere, corresponding to rotational positions that give known distances of the focal point 80 with respect to the distal end 110 of the stand. These could, in a preferred embodiment, correspond to placing the focal point at various discrete distances above or below the plane defined by distal end 110. Instead of a spiral groove, a straight groove with selectable pin slots, or with depth indicators plus a set screw, could be used to allow depth selection. With such depth selection, it is possible to perform several procedures at a site with a single device, for example to make an incision in tissue for a medical purpose and then cauterize the operating area. It would also be possible to make the movement of the focal point with respect to the end 110 of the stand be a continuously variable property, by providing additional parts to the housing/stand system allowing them to be locked at any relative distance. For example, setscrews are among many mechanisms that can produce that effect.

Another means for varying the focus of the laser beam is to put one or more adjustable lenses into the optical path, for example just below cylindrical lens 50, in the general manner shown in FIG. 3.

The functional purpose of the stand 100, if provided, is to standardize the distance from a lens such as lens 50 to the tissue. The stand 100 can be made of any suitable material. It may be solid and opaque. More preferably, it is solid and transparent to visible light, but not transparent to laser frequencies in the 1300-1700 nm range. Thus, any illuminating visible light (whether from the outside or emitted by the device) can be seen, allowing the progress of the procedure to be monitored, while the laser energy can be confined to the inside of the stand. Alternatively, the stand is not solid, but serves only to position a tissue-contacting ring or several feet on the tissue, with a few rods or other connectors holding a ring or feet in place with respect to the housing 20. Even one positioned foot can be enough to establish the distance between the diode and the treatment site.

### Wavelength

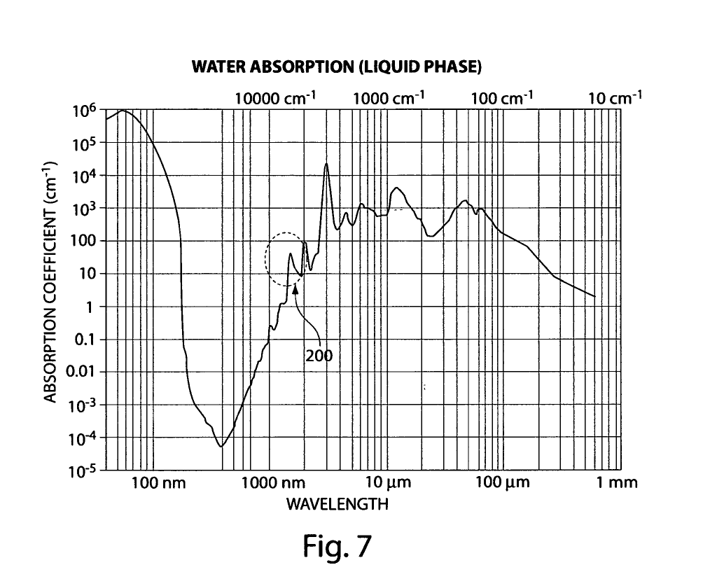

FIG. 7 shows the “1/e” extinction depth of visible and infrared light in water as a function of wavelength. A preferred emission wavelength band is generally in the range of about 1300-1700, preferably about 1350-1600 nm, more preferably 1400-1550 nm, falling in a region of local maximum water absorption. The local water peak 200 (circled with dashes) has an absorption coefficient greater than 10/cm (i.e., is attenuated by 1/e to the tenth power per cm of penetration) and includes wavelengths in a band from about 1400 to 1550 nm. This band of wavelengths has the useful property of being readily absorbed by essentially all soft tissue, and the critical property of being significantly absorbed by replaceable tissues of the eye, including the aqueous and vitreous humors and the lens and cornea, before damaging the retina, even at power levels sufficient to cut or cauterize tissue. (In short, radiation at these wavelengths is “eye safe.”)

A secondary eye safe band is found in the region of about 1800 to 2500 nm. In this band, the radiation is also absorbed in the eye before reaching the retina, but the tissue penetrating depth rapidly gets shorter, leading to greater difficulty in incising or excising tissue, and to limitation of coagulation depth in cauterization.

A preferred laser diode for the purposes of the invention is an InGaAsP-based system, emitting in the 1300-1610 nm region, as described in our co-pending application U.S. Ser. No. 11/233494, published as US 2007/0002915. Other systems using semiconductor laser diodes emitting in this region may also be useful. The key feature of the preferred wavelength of the laser is that on one hand it has a long enough wavelength to emit in or near the “water peak” beginning at about 1400 nm. The important aspect of the water peak is that in these wavelengths, a laser beam in water is attenuated by a factor of 1/e (1 divided by 2.7, or about 0.37) per millimeter, or greater. So incoming light in this band is attenuated by at least e exp 10 (ca. 20,000) per cm (i.e., per 10 mm) in the eye, before getting to the retina. Since the eye is at least 3 cm deep from the cornea to the critical areas of the retina, such as the fovea, an attenuation of at least about 8×(10 exp 12), about 8 trillion, is obtained in this wavelength range.

Because the lens of the eye will focus a beam spread over its effective surface (about 100 mm sq., when pupils are dilated) onto a small spot on the retina (for example, about 10 micron sq), it can concentrate radiation by a factor of up to about a million; but there will still be an attenuation of laser beam intensity of greater than 8 million during passage from the cornea to a spot on the retina.

Such an attenuation can be used as a functional definition of “eye safe”: a laser beam is eye-safe when, although damage may be done to the cornea, lens and vitreous by exposure to a beam, the non-replaceable component of the eye, the retina, will almost certainly be spared.

This is not to imply that operators of this laser system need not wear eye protection. Appropriate eye protection should be worn by all personnel and the patient. However, in emergency and field conditions there is less control over entry of persons into the area in which the laser will be used for coagulation, cautery, tissue cutting and the like. Under such conditions, the use of a laser system which is inherently less damaging to the eye is an important safety precaution. Attenuation is also an important safety consideration even when the device is used in a conventional medical facility, as a safeguard in the event of accidental eye exposure to the laser beam.

### Laser Mounting

The InGaAsP semiconductor diode laser that is presently preferred for this application is a physically small device, but because of its power, careful mounting and heat management measures are required. The laser power of the device and its pulse rate needs to be engineered for the application of interest. Optical powers of greater than 1 watt and preferably greater than 3 watts are needed to provide sufficient heating power to the tissue. Moreover, in order to avoid burning the tissue, it is preferable to pulse the laser so that appropriate heat is conveyed to the tissue without burning. The pulse duration and frequency, and the length of time that the beam is applied to a given region of tissue, will be made proportional to the power delivered and to the depth and extent of the selected treatment.

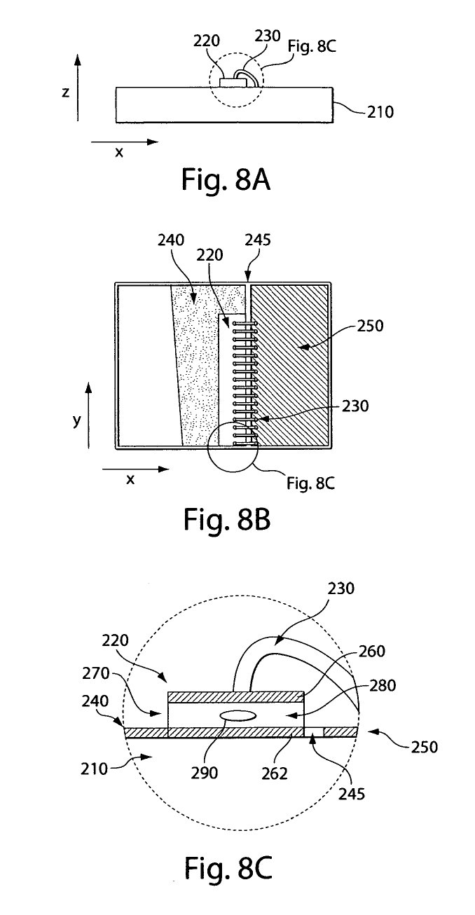

Because a laser output power of several watts can require the generation of five watts or more of heat inside the semiconductor laser, heat management is an important concern and drives some aspects of the packaging. FIG. 8 shows the general structure of a single diode laser for use in the coagulating device of the invention. FIG. 8A is a face-on view of the laser chip, showing a substrate 210 for the chip, for example BeO or AlN (aluminum nitride), which are good heat conductors but electrical insulators. The laser diode 220 sits on the electrically insulating substrate 210. A first electrical connection 230 connects the top of laser diode 220 to a conductor, such as first solder layer 250.

FIG. 8B shows the device of FIG. 8A in top-down view. The conductor 230 is one of several (16 in this illustration), connecting a first solder layer 250 to the top of the diode 220, which is conducting. The diode chip 220 sits on a second solder layer 240, which covers some or all of the rest of the top of the substrate 210. The two solder layers, which function as electrodes for the laser diode, are separated by an insulating gap 245, which optionally contains a non-conducting material.

FIG. 8C shows a schematic enlargement A of the laser diode chip, in the end view of FIG. 8A. The substrate 210 is shown, as well as the wire(s) 230, the solder layers 240 and 250, and the gap 245. The diode 220 is shown as having conductive layers 260, 262 for interfacing with wire 230 and solder layer 240. The semiconductor layer 280 has reflecting sides 270. The laser beam is emitted from the center region 290 of the diode 280. The emitted beam is often elliptical in profile. The ellipticity of the beam profile may be corrected with a cylindrical lens or other correcting device, which is not illustrated here, but is represented as a lens 50 in FIG. 1, for example.

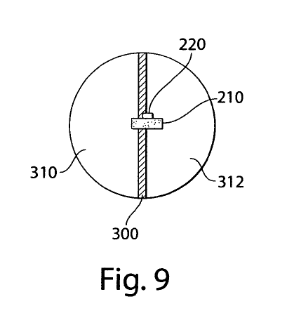

These devices are small. In FIG. 8A and 8B, the dimensions x, y, z of an actual device are about 4 mm, 3 mm and 0.8 mm. To remove heat from this small area, heat sinks are required, in addition to in-chip heat sinks such as substrate 210. FIG. 9 shows the substrate 210 and laser diode 220 of FIG. 8 at about twice actual size. The diode is surrounded by heat sinks 310, 312, which are separated by an electrically insulating layer 300. In this embodiment, the heat sinks are also conductors, and replace the wires 230 of FIG. 8. Electrical contacts are provided to the heat sinks (not shown) for connection to a battery or other power source. The assembly of FIG. 9 could, as laser assembly 40, fill part or all of the inside of the housing 20, as shown in FIGS. 1-6.

In addition, the heat sinks 310, 312 might have passages through them to promote heat exchange between the heat sinks and the air, or other fluid. The heat sinks 310 and 312 could also have an expanded surface area to facilitate the transfer of heat to a fluid, such as air or liquid. These could take any of a variety of shapes and structures designed to maximize heat transfer to a fluid, including without limitation finned protrusions or ribs, with or without fenestrations; meshes and expanded meshes; and other known heat transfer systems. There could also be a fluid circulation system for circulating a heat transfer fluid, such as saline, or air, or other suitable fluid, through and/or past the heat sink to improve heat removal and minimize the volume and weight of heat removal systems. Circulation could be one pass or recirculating.

### Additional Features

Various added features can improve the usefulness of the diode laser coagulator. A timer, to operate the diode laser for a predetermined interval, could be useful. A first area could be treated and then the device could be moved to a second area, or to another patient. A visual indicator that the laser is operating would be useful, for example a visible-light emitting diode powered by energy derived from the infrared laser beam. As mentioned above, a visible light source might be included with the device—perhaps also from diodes, for energy efficiency—to illuminate the target region, and optionally the region around the device as well.

Any of these ancillary devices, or a power-management function, or a timing function, could be supplied in an on-board control package, schematically listed as 45 in FIG. 4; or, in a disposable device, the electronics could be placed in a non-disposable part of the instrument, confining the portion disposed of after use to as little of the overall device as is possible.

### Use of the Device

The resulting instrument has a simplicity of operation and use that is highly suitable for field and emergency conditions. The operator points the device at a wound in need of cauterization and presses a button. Since the distance between the device and the site of operation is a few inches or less, elaborate aiming routines are not required.

Cauterization of the wound, when exposed to the laser beam, will typically require less than a minute. Beam location can be judged by watching the site, with the aid of an visible light source, and observing where coagulation is beginning.

In cauterization, and many other procedures, the ability to control the rate of coagulation or other treatment by adjusting either the distance of the laser from the target, or the input power to the laser, is very helpful for avoiding unwanted side effects such as charring, and transmission of energy deeper into tissue than is desired.

Notably, the need for eye protection is minimized, and the consequences of failure of eye protection are less severe, because the wavelength ensures that brief eye exposure to the beam will not damage the retina. The beam may produce cataracts, if sufficiently strong, and it is advisable for operators to wear appropriate goggles to filter out the 1350-1600 band. For example, lenses filled with water, optionally in the form of a gel, would be suitable. However, because the beam is diverging after reflecting in part from the patient tissue, the intensity drops with increasing propagation distance, typically in proportion to the inverse square of the distance, and so bystanders not close to the site of application of the beam are both safe from retinal damage, and are at relatively low risk of damage from cataract or other external damage.

Even in a non-emergency situation, a semiconductor diode laser operating in the 1300-1700 water absorption peak has significant advantages over a non-laser source of energy, such as electrocautery. The laser system, because the energy is specifically absorbed by tissue, can cauterize without emitting smoke or toxic vapors, or creating eschar (a post-burn carbonized scab). There is no need to route electricity through the body, and no RF static, and so there is less interference with medical electronics and implants. A further advantage of the device of the invention is that the treatment need not involve contact of the instrument with the patient's tissue. This can be advantageous in emergency or field conditions, where it may not be feasible to sterilize the patient's skin, or the device. Moreover, the lack of contact eliminates the risk of transferring undesirable cells, or microorganisms, between different sites in a patient during a procedure.

The system of the invention opens up additional opportunities for minimally invasive surgery. Because the individual diodes of the semiconductor laser are small—only a few mm on a side—it is possible to mount the diodes on an endoscope or similar device, or even on a catheter, so that energy can be delivered via minimally invasive routes to tissues in many interior locations in the body. Applications available for a semiconductor diode laser with good tissue absorption include many possible operations. One application is cardiac ablation, for example, in the Maze procedure for fibrillation. Another application is tumor ablation, especially in ducted organs such as the bladder, the prostate, the uterus, the breast, the ovary and the kidney. For example, treatment of ductal carcinoma in situ of the breast would be feasible with the device of the invention, with minimal damage to nearby normal tissue. Catheters carrying lasers can be passed via the bloodstream to treat aneurysms and blockages of the circulation, and to treat tumors lying near arteries, for example in the brain. In internal applications, different provisions for cooling of the laser diode may need to be made, which may include circulation of external fluids, such as isotonic saline, around the diode, but not in the path of its emitted laser beam, if the fluid contains water. The diode may be provided with heat exchange means adapted from those described above.

As an example, in the treatment of endometriosis, a thin layer of cells must be removed or killed without penetrating or killing the underlying tissue wall. A relatively uniform laser beam with a high water absorption coefficient, for example as in FIG. 1 or 4, can selectively kill cells at the surface, and has a very low risk of penetration of the tissue wall.

Unless defined otherwise, all technical and scientific terms used herein have the same meanings as commonly understood by one of skill in the art to which the disclosed invention belongs. Although any methods and materials similar or equivalent to those described herein can be used in the practice or testing of the present invention, the preferred methods, devices, and materials are as described. In the present application, “cut” or “cutting” includes excision and incision. Absence of a particular use in a list of uses does not exclude that use from the set of uses contemplated for the invention. Publications cited herein and the material for which they are cited are specifically incorporated by reference, where such incorporation is permitted. Nothing herein is to be construed as an admission that the invention is not entitled to antedate such disclosure by virtue of prior invention, where relevant. Those skilled in the art will recognize, or be able to ascertain using no more than routine experimentation, many equivalents to the specific embodiments of the invention described herein. Such equivalents are intended to be encompassed by the following claims.

1. A portable semiconductor diode laser system for medical or surgical treatment, the system comprising:
at least one semiconductor laser diode emitting in the infrared between about 1300 and 1700 nm, to administer therapeutic doses of laser energy to a patient; and
a power supply to drive said diode;
wherein the infrared emission is conducted from the emitting diode to the site of treatment at least partially through space.

2. The system of claim 1 wherein the laser diode is an InGaAsP laser diode.

3. The system of claim 1 wherein the power supply is provided by one or more of a rechargeable battery, a non-rechargeable battery, a wired connection to a low voltage power source, and a wired connection to a high voltage power source.

4. The system of claim 1 wherein the treatment is selected from cauterization, coagulation, ablation, cutting, tissue heating, treatment of tissue surfaces, and treatment of skin disease.

5. The system of claim 1 further comprising an aiming system using visible light to assist the operator in directing the infrared laser beam to the site of treatment.

6. The system of claim 1 further comprising a stand to position the diode laser source at an appropriate distance from the patient.

7. The system of claim 6 wherein the stand maintains a fixed distance between the laser source and the site of operation on the patient.

8. The system of claim 6 wherein the stand includes two or more settings that provide two or more preselected distances between the laser source and the site of operation on the patient.

9. The system of claim 6 wherein the distance between the laser and the patient is varied continuously by adjustment of the stand.

10. The system of claim 9 wherein the adjustment operates as a zoom lens.

11. The system of claim 1 wherein the laser beam comes to a focus due to the action of a fixed lens acting on the output of the diode laser.

12. The system of claim 11 where the locus of the focal point is adjusted by at least one of an adjustment of a stand, and an adjustment of an optical focusing means.

13. The system of claim 1 wherein the laser diode is mounted on an endoscopic device or a catheter.

14. The system of claim 13 wherein the laser diode mounted on a catheter or endoscopic device is capable of being moved through a natural body passage or opening to reach a site of operation.

15. The system of claim 14 wherein the natural body passage is selected from one or more of an artery, a vein, a nasal passage, the alimentary and gastrointestinal tracts, the pulmonary tract, the milk ducts, the urinary tract, the female reproductive tract and the male reproductive tract.

16. The system of claim 1 wherein the wavelength is in the range of 1350-1600 nm.

17. The system of claim 1 wherein the wavelength is in the range of 1400-1550 nm.

18. The system of claim 1 wherein the semiconductor diode laser is mounted in a heat sink.

19. The system of claim 18 wherein the heat sink has at least one of an expanded surface area and a fluid circulating means for improving heat transfer.

20. The system of claim 1 wherein the emitted optical power is greater than 1 watt.

21. The system of claim 1 wherein the laser is pulsed during each treatment cycle.

22. The use of the system of claim 1 for conducting at least one medical procedure selected from the cauterization of wounds, the ablation of tissue, the coagulation of blood or other fluid, the cutting of tissue, the local heating of tissue, the treatment of tissue surfaces, and the treatment of skin diseases.

23. A method for conducting, at a tissue site, at least one procedure selected from the cauterization of wounds, the coagulation of fluids, the ablation of tissue, the coagulation of blood or other fluid, the cutting of tissue, the local heating of tissue, the treatment of tissue surfaces, and the treatment of skin diseases, the method comprising:
providing at least one portable semiconductor diode laser operating in the 1300-1700 nm wavelength band;
placing said laser at a location where the beam output by said laser will impinge on the tissue site to be treated by said procedure; providing a power source for said laser;
providing at least one lens to control the divergence of the laser beam; and
activating said diode laser for sufficient time to conduct said selected procedure.

24. The method of claim 23, wherein the propagation of the laser beam from the laser to the tissue site occurs at least in part without the use of an intermediate fiber optic beam carrier.

25. The method of claim 23 wherein the lens or lenses are arranged so that the laser beam has a focal point.

26. The method of claim 23 wherein the portable semiconductor diode laser operates in the range of about 1350 nm to about 1600 nm.

27. The method of claim 23 wherein the portable semiconductor diode laser operates in the range of about 1400 nm to about 1550 nm.

28. The method of claim 23 wherein the portable semiconductor diode laser power source is a local battery.

29. The method of claim 23 wherein the portable semiconductor diode laser power source is located remotely from the laser.

30. The method of claim 23 wherein the tissue site is on the exterior of the body.

31. The method of claim 23 wherein the portable semiconductor diode laser is carried on an endoscope or catheter.

32. The method of claim 31 wherein the tissue site is accessed through a natural body passage or opening to reach a site of operation.

33. The method of claim 32 wherein the natural body passage is selected from one or more of an artery, a vein, a nasal passage, the alimentary and gastrointestinal tracts, the pulmonary tract, the milk ducts, the urinary tract, the female reproductive tract and the male reproductive tract.

34. The method of claim 23 wherein the emitted optical power is greater than 1 watt.

35. The method of claim 23 wherein the laser is pulsed during each treatment cycle.

36. The method of claim 23 wherein the laser is a semiconductor diode laser.

37. The method of claim 23 wherein the laser is a In/Ge/As/P laser.

38. The method of claim 23 where the device further comprises a heat sink in contact with a fluid.

39. (canceled)
